(19) United States
(12) Patent Application Publication
(10) Pub. No.: US 2008/0051886 A1
(43) Pub. Date: Feb. 28, 2008

(54) Method and device for vision correction via dual-optics accommodating intraocular lens

(76) Inventor: J.T. Lin, Oviedo, FL (US)

Correspondence Address: J. T. Lin, 4532 Old Carriage Trail, Oviedo, FL, 32765, US

(21) Appl. No.: 11/508,877

(22) Filed: Aug. 24, 2006

(51) Int. Cl. A61F 2/16

(52) U.S. Cl. 623/6.34; 623/6.37

(57) ABSTRACT

Method and design of a dual-optics accommodating intraocular lens (IOL) for vision correction of adult and pediatric eyes after cataract surgeries are disclosed. For adult eyes, a positive accommodation amplitude greater than 3.5 diopter and preferably 4.0 to 10.0 diopter may be achieved by optimal configurations having the positive-power front-optics moves toward the cornea, whereas the negative power back-optics moves in the opposite direction. In contrast, a negative accommodation is required for pediatric eyes and may be achieved by a reversed configurations. The enhanced efficiency, up to 500%, is proposed by preferred embodiments based on new lens design formulas and calculation steps for the IOL power pre-determined by the measured ocular parameters including the corneal power, IOL position and the vitreous cavity length of the eye.

### BACKGROUND OF THE INVENTION

1. Field of the Invention

The present invention relates to intraocular lens (IOL) and more particularly, to provide positive (negative) accommodation for adult (pediatric) eyes after cataract surgeries.

2. Prior Art

Intraocular lenses (IOL) have been used for vision corrections particularly for patients after cataract surgery. For aged eyes, accommodation is required in order to see both far and near after the basic correction for far vision. Accommodation may be achieved by allowing the implanted IOL to move along the axial axis of the eye.

Various accommodating IOLs (AIOL) have been studied including single-optics and dual-optics configurations such as the prior arts of U.S. Pat. No. 5,275,623 (Sarfarazi); U.S. Pat. Nos. 5,476,514, 5,496,366, 5,674,282, 6,197,059 (Cumming); U.S. Pat. No. 6,302,911 (Hanna); U.S. Pat. Nos. 6,261,321 and 6,241,777 (Kellan); U.S. Pat. No. 6,616,691 (Tran); U.S. Pat. No. 6,660,035 (Lang); and US Pat. Application No. 2003/0204256 (Peng); 2003/0187504 (Weinschenk and Zhang); WIPO 2000/66037 (Glick et al).

The accommodating efficiency in single-optics defined by an M-function M=A/(dS), or the accommodating amplitude (A) per 1.0 mm of the AIOL forward movement (dS) had been derived analytically (Lin, J. Refract Surg. 2005; 21:200-201) and by raytracing methods. However, no analytic formulas are available for dual-optics AIOL other than the numerical analysis of raytracing by Ho et al (J. Cataract Refract Surg. 2006; 32:129-136) which was also limited to the simple cases that only one of the dual-optics is mobile. In practice, the ciliary body contraction would cause both of the optics to move in either direction, toward the cornea or the retina. The existing AIOL designs with about 2.5 diopters or less accommodation amplitude (for a typical 2.0 mm axial movement) could only marginally meet the clinical need, if one considers the 50% comfortable level. Efforts for higher accommodation have been proposed in a suspension structure (by Lang et al in U.S. Pat. No. 6,660,035) and in a secondary-lens structure by Ben-nun and Alio (J. Cataract Refract Surg. 2005; 31:1802-1808).

Most of the prior arts (Cumming, Hanna. Lang, et al) are limited to single-optics AIOLs which suffer a rather low accommodation amplitude (A<2.5 diopter). The prior arts in US Pat. of Tran (U.S. Pat. No. 6,616,691) and US Pat. Applications of Peng and Weinschenk et al (2003/0204256, 2003/0187504) proposed the dual-optics AIOL which, however, are limited to the movement of the front-optics and therefore its accommodation (A) values are also limited to a low value (say <3.0 diopter). Moreover, these prior arts estimated the A value based on a wrong formula which underestimates the A value about 50%. Due to the lack of accurate analysis and new formulas, all the existing IOL designs suffered a limited improvement of the accommodation.

Therefore, a need continues to exist for a more efficient AIOL (having A>3.5 diopters) based on new concepts, analysis and new designs to be disclosed in the present invention. Furthermore, all the AIOLs in prior arts are designed for adult eyes after cataract surgery which requires a positive (A>0) accommodation to see near. In contrast, for eyes after pediatric cataract surgery a negative accommodation (A<0) is required to compensate the myopic-shift resulting from the continuing growth of the axial length. AIOLs with negative A values have never been explored in the prior arts.

### BRIEF DESCRIPTION OF THE INVENTION

The present invention is to design dual-optics AIOL based on new configurations and analysis which allow either or both of the optics to move in forward or backward directions along the axial axis. Accordingly, one objective of the present invention is to provide new formulas and methods which could be used to significantly improve the accommodation.

Another objective is to define the optimal AIOL power and moving directions for maximum accommodation amplitude (A).

Still another objective is to define the optimal design for positive accommodation (A>0) for adult eyes after cataract surgery, where near vision may be accommodated.

Still another objective is to define the optimal design for negative accommodation (A<0) for pediatric eyes after cataract surgery, where the myopic-shift due to axial (globe) growth may be accommodated or compensated by AIOL.

Still another objective is to provide systematic design steps using the measured ocular parameters to pre-determine the basic correction power which further defines the optimal front and back optics power of the dual-optics AIOL.

### BRIEF DESCRIPTION OF THE DRAWINGS

FIG. 1 shows the schematics of the relative positions of the cornea and dual AIOL.

FIG. 2 shows the AIOL configurations and moving directions for maximum positive accommodation.

FIG. 3 shows the AIOL configurations and moving directions for maximum negative accommodation.

FIG. 4 shows the enhancement factor based on the new design comparing to prior arts.

FIG. 5 shows the flowchart for AIOL design for positive and negative accommodation.

FIG. 6 shows the side-view of the preferred AIOL embodiment implanted in the posterior chamber.

FIGS. 7A and 7B show the relative preferred movements of the optics with respect to the ciliary body contraction for different optics diameters.

### DETAILED DESCRIPTION OF THE INVENTION

### The New Formulas

TABLE 1
Formulas and definitions for dual-optics accommodating IOL
(Lin, in Mastering IOLs: Principles and Innovations. New
Delhi, Jaypee Brothers Pub. 2006).

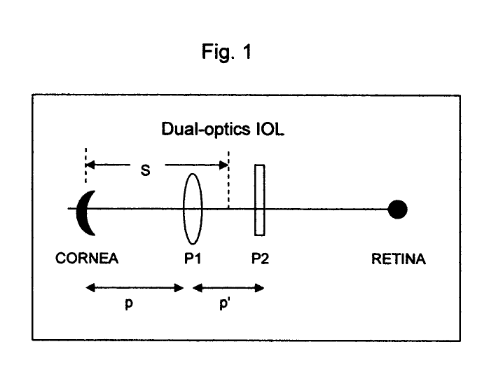

A = M(dS1) + M′(dS2)
M = (P1/P)Mo − C
M′ = (P2/P)Mo′ − C
Mo = 1336(1/F² − Z2/fc²)
Mo′ = 1336(Z1/F² − 1/fc²)
C = Z²(P1P2/1336)
Zj = 1 − (p′/1336)Pj, j = 1, 2
Z = 1 − S/fc, S = p + p′(P2/P)
where (referred to FIG. 1):
A = accommodation amplitude (for both optics are mobile)
dSj = movement of the front-optics (j = 1), back-optics (j = 2)
(P1, P2) = power of the (front, back)optics
P = P1 + Z1P2, total power of the dual-optics IOL (P = 1336/F).
p = distance of the front-optics and cornea
p′ = distance of the front-optics and back-optics
S = effective position of the front-optics
fc = effective focal length of the cornea related to its power by
fc = 1336/Dc.
P = P2 + (Z2)P1 = P1 + (Z1)P2

As shown in Table 1, the accommodating amplitude (A) of a dual-optics AIOL is given by two components

A=M(dS1)+M′(dS2), (1)

where the M-functions represent the increase of A per 1.0 mm forward movement of the optics. The movement directions are defined as forward (for dSj>0) or backward (for dSj<0). Detail derivation of formulas in Table 1 is shown elsewhere ((Lin, in Mastering IOLs: Principles and Innovations. New Delhi, Jaypee Brothers Pub. 2006).

Since both M and M′ are proportional to the power of the moving optics (P1 or P2), greater positive A may be achieved by combining a positive optics (P1>0) and a negative (P2<0) moving in opposite directions, or dS1>0 (front-optics moves forward toward the cornea) and dS2<0 (back-optics moves away from the cornea). The general rules based on the above new formulas are: positive-power optics with forward movement, or negative-power optics with backward movement produces positive A (or myopic-shift of the image for near view) which is required in adult eyes after cataract surgery. On the other hand, negative A (or hyperopic shift) is needed for pediatric eyes after cataract surgery, where the axial elongation (growth) of the eye results myopia.

For typical values of S=5.0 mm, and corneal power Dc=43 D (‘D” stands for diopter), Z=0.84, Eq. (1) may be further simplified as

A=0.054(1+0.01P)B−C(dS1+dS2), (2.a)

B=P(dS1)−P2(Z1dS1−dS2). (2.b)

where the total (basic) IOL power P=P1+(Z1)P2. For maximum A, the above new formulas of Eq. (2) readily tell us that one needs a maximum B value which could be achieved by a negative P2 and a positive P1 having dS1>0, dS2<0, for adult eyes with positive accommodation (A>0).

In contrast, A<0 for pediatric eyes would prefer a positive P2 (with dS2<0) and negative P1 (with dS1>0) for maximum amplitude (or absolute value of A) of the negative accommodation.

### Enhancement Analysis

One may define an enhancement factor E=A*/Ao, with A* being the maximum of A, when dS2=−dS1; and Ao being the minimum, when dS1=dS2 in dual-optics, or the single-optics IOL case with P2=0. Using this definition, Eq. (2) allows us to easily derive

E=1−(P2/P)(dp′/dS1). (3)

where dp′=dS1−dS2 is the net separation change of the dual optics. In order to have E greater than 1.0 for A>0 case, one requires P2<0, dp′>0, dS1>0, P>0, and the back-optics moves backward while the front-optics moves forward. For example, for a basic plus IOL power of P=20 D, and dp′=2dS1 (or dS2=−dS1) with dS1=1.0 mm, E=250% and 500%, respectively, for P2=−15 and −30 D; and the associate front optics power of P1=+35 and +60 D.

In contrast, for negative A, the preferred conditions for enhanced A (or higher negative value of E) are: P>0, P2>0 (with dS2<0), dp′>0, but P1<0 (with dS1>0).

The above new findings with enhancement factor (for A>0 case) as high as 500% are not available in the prior arts based on single-optics AIOL. The advantage of enhanced accommodation in dual optics, dual-mobile design by optimal P1 and P2 and their moving directions has not been disclosed in any of the prior arts. The new formulas of the present invention provide the basis of the new AIOL designs is not available in any of the prior arts.

### The Preferred Configurations

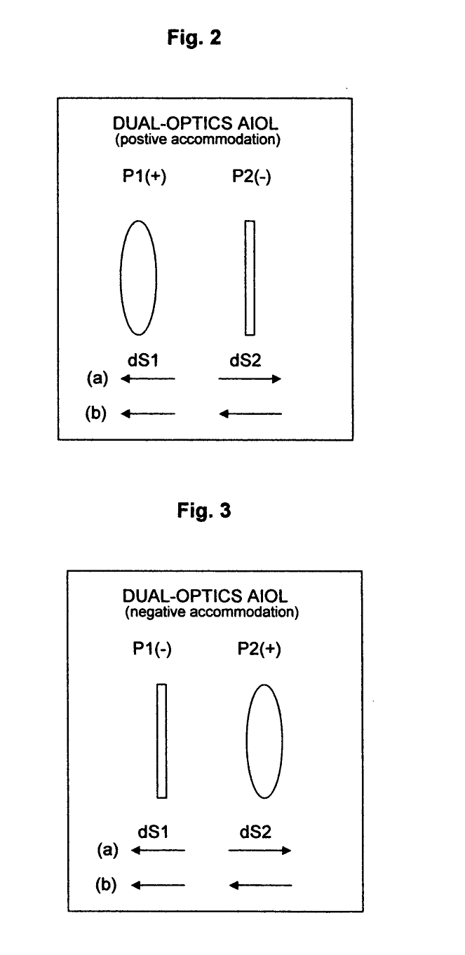

FIG. 2 shows the schematics of dual-optics AIOL having a positive accommodation (A>0), for case (a) maximum accommodation having the front positive-power optics (P1>0) moving toward the cornea, while the back negative-power optics (P2<0) moves toward the retina. In comparison, case (b) having both optics moving in the same direction shows a smaller accommodation as analyzed by the new formulas.

FIG. 3 shows the AIOL having a negative accommodation (A<0), where case (a) with the 2 optics moving in opposite directions is more efficient than case (b) having the same directions.

Therefore the preferred movement directions of the present invention (as shown by FIG. 2) include that the front-optics moves forward (to the cornea or dS1>0) and the back-optics is immobile (dS2=0) or moves backward (away from the cornea, or dS2<0), where the most preferred parameters are dS1>1.0 mm and dS2 about 0.5 mm, such that the separation change dp′>1.5 mm, or the associate A>3.5 D. Other preferred examples are calculated as follows based on Eq. (2). For P=20 D, dS1=1.5 mm, dS2=−0.5 mm, one may obtain A=4.1, 5.2 and 8.2 D, for P2=−20, −30 and −40 D, respectively for positive accommodation, comparing to a much lower A=1.3 D in single-optics IOL (for P2=0). On the other hand, for negative accommodation, A=−1.2 and −4.7 D, for P2=40 and 60 D, respectively.

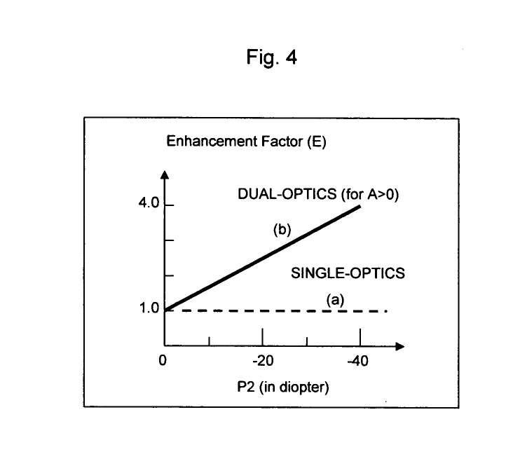

FIG. 4 shows the enhancement factor (E) being an increasing function of the back-optics power (P2) for the case of positive accommodation. The E=1.0 presents the conventional single-optics performance, or dual-optics having P2=0 and P1=P. It should be noted that, for a given basic correction power (or the total power) P=P1+(Z1)P2, the E-function is linearly proportional to (−P2) and requires P2<0 for higher E.

Accordingly, the preferred embodiment of the present invention includes an AIOL having a basic correction power P=15 to 25 diopter (D) which is the refraction error of an eye after cataract surgery, or the power needed for emmetropia.

For positive A (used in adult eyes): a back-optics having a negative power (P2<0), preferred P2 of −2 to −40 D, and preferred P2 of −10 D to −20 D, and a front-optics having a power given by P1=(P−P2)/(Z2) which is about +15 D to +60 D, and preferably about +30 D to +40 D. The configurations of the front-optics include bi-convex, convex-piano or convex-concave, whereas the back-optics is bi-concave, concave-piano or concave-convex. Under the above preferred embodiments, the preferred accommodation amplitude is about +3.5 D to +10 D, or preferably about +4 D to +10 D for a typical axial movement or separation change (dp′) of the AIOL about 2.0 mm resulting from the ciliary body contraction.

For negative A (used for pediatric eyes), the preferred parameters are: a back-optics having a positive power (P2>0), preferred P2 of +15 to +60 D, and preferred P2 of +20 D to +40 D, and a front-optics having a power given by P1=(P−P2)/(Z2) which is about −40 D to −5 D, and preferably about −10 D to −30 D. The configurations of the back-optics include bi-convex, convex-plano or convex-concave, whereas the front-optics is biconcave, concave-plano or concave-convex. Under the above preferred embodiments, the preferred accommodation amplitude is about −3 D to −10 D, or preferably about −4 D to −6 D for a typical axial movement or separation change (dp′) of the AIOL about 2.0 mm resulting from the ciliary body contraction.

The AIOL optics preferable are made from a suitable flexible polymer such as silicone, or stiff plastic such as polymethylmethacrylate (PMMA) and have diameters of about 4 mm to 7 mm, and most preferable about 5 mm. It also consists of a pair of haptics located on sides of the optics and the contact plates configured to complement the inner peripheral region of the capsular bag.

### The Basic Correction Power (P)

Due to the difficulty of measuring the accurate refractive error (the basic power P) of an eye postcataract surgery, P could only be estimated by the available measured parameters of the corneal power (Dc), posterior vitreous length or position of the lens capsule (X) and the estimated lens position of the front optics (p). Given the known parameters of p′ (about 0.5 to 1.0 mm), p (about 4 to 5 mm), Dc (about 40 to 45 D), the required front-optics power (P1) for emmetropia may be calculated from yet another new formula developed by Lin (in Mastering IOLs: Principles and Innovations. New Delhi, Jaypee Brothers Pub. 2006).

P1=(Po−Dc/Z−P2)/Z2, (4)

where Po=1336/X, defined by the measured vitreous cavity or natural lens position prior to the cataract surgery. The calculated P1 and the pre-determined P2 then allow us to calculate the basic correction power by

P=P1+(Z1)P2. (5)

### Error Analysis

For the actual AIOL designs, it is critical to have the error analysis of each of the ocular and AIOL parameters. The dominant error source of P1 in Eq. (4) is the front-optics position (p) which could only be estimated pre-AIOL based on the position of the natural lens capsule before the cataract surgery. A typical error of p is about 0.3 to 0.5 mm. The second error source of P1 is from the true corneal power (Dc) having an accuracy limited by its posterior surface curvature, although its front surface power may be accurately measured by kerometry device. The third error source is due to the shrinkage of the capsular bag upon healing. Given these error sources, one should expect an approximated error of P1 about 0.5 to 1.0 diopters which can not be eliminated by the existing ultrasound technology. They also pre-exist by the AIOL position error which could only be estimated pre-AIOL implant.

The conventional and the existing AIOL devices providing an accommodation amplitude (A) about 2.0 to 2.5 diopters, therefore, is not clinically practical, if one includes the error of about 0.8 diopter and only 50% of A could be used under a typical comfortable ciliary body contraction level of a presbyopic eye. The enhanced A value of about 3.5 to 10.0 diopter achieved by the optimal design of the present invention overcomes the above described drawbacks of the prior arts.

### Detailed Description of Lens Design

It is important to note that formulas of Eqs. (4) and (5) are the critical elements in determining the optimal design parameters of the new AIOL. The enhancement factor (E) of Eq. (3) and the A value defined by Eq. (2) require the knowledge of the basic power (P) for a given manufacturer-defined power (P2). Therefore, Eqs. (4) and (5) are essential for clinically useful AIOL designs. All the existing prior arts failed to define the key parameters (P1, P2 and P) due to the lack of the lens design equations as disclosed in the present invention. The over-simplified 1-optics formulas used by prior arts such as US Pat. Appl. No. 2003/0187504 and 2003/0204256 could not provide any of the new findings disclosed in the present invention. The above prior arts also suffer errors over 50% for the estimated accommodation amplitude.

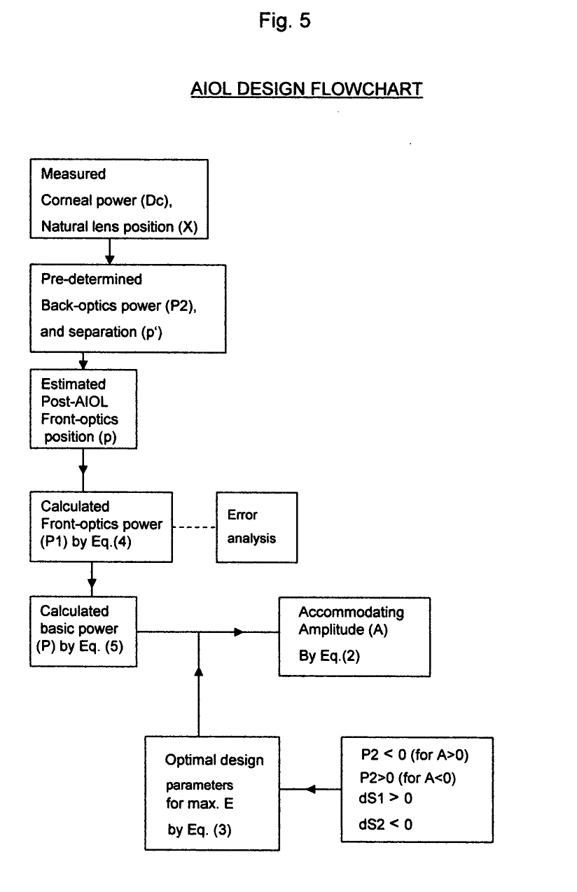

FIG. 5 summarizes the flowchart (steps) of the AIOL design which combines the measured parameters and the new formulas to calculate (predict) the resulting accommodating amplitude (A) and the enhancement factor (E), for both positive (A>0) and negative (A<0) accommodations governed by P2<0 (P1>0) and P2>0 (P1<0), respectively.

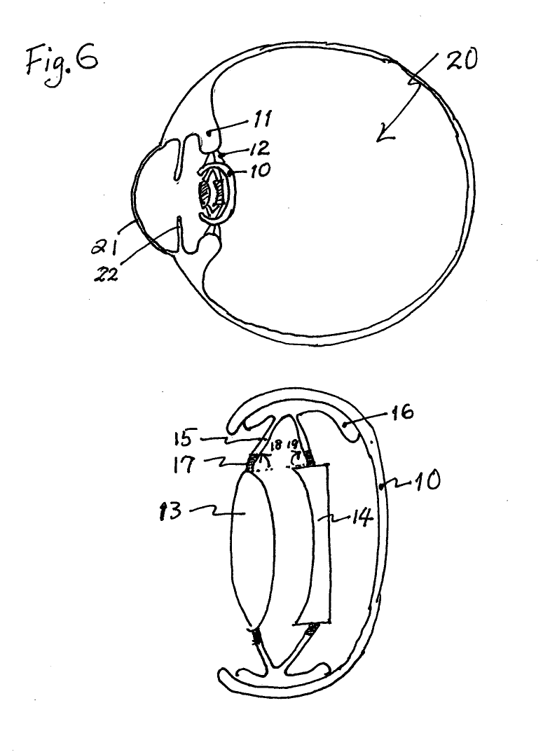

FIG. 6 shows the preferred embodiment of the AIOL (a side view) implanted in the posterior chamber defined by the lens capsular bag 10 connected to the ciliary body 11 by the zonules 12. Also shown are the iris 21 and cornea 22 of the eyeball 20. The AIOL consists of the front optics 13, back optics 14, haptics 15 that are shaped to fill the equatorial region 16 of the capsular bag 10, and the hinge portions 17 at each of the optics-hapics junction. When the ciliary body 11 contracts, the angles 18 and 19 of the hinge portions 17 decrease and causes the dual optics 13 and 14 to move in the opposite directions, that is, the spacing (p′) increases and results the maximum accommodation of the eye based on the new formulas.

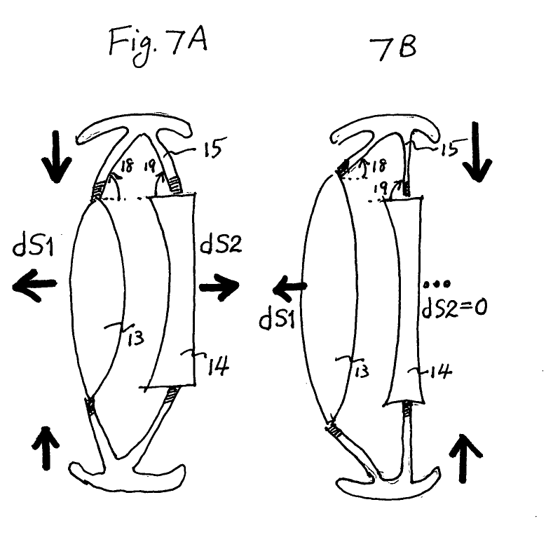

As shown in FIG. 7A, the movement amounts (dS1 and dS2) could be comparable when the two optics 13 and 14 have approximately same diameters, whereas the front-optics 13 could move more when its diameter is slightly larger than that of the back optics 14. Depending on the hinge angles 18 and 19, the back-optics may be immobile (shown by FIG. 7B with a right angle of 19 and dS2=0), while the front-optics has a maximum movement.

Greater details of the haptics and hinges are available in prior arts such as U.S. Pat. Nos. 5,476,514 and 6,302,911 and the commercial AIOLs made by HumanOptics (Germany) and C&C Vision (USA).

While the present invention has been described by various specific examples and embodiments, it is to be understood that the invention is not limited thereto and that it can be variously practiced within the following claims.

I claim:

1. An intraocular lens system for replacement of a natural lens of an eye during eye surgery, consisting of:
(a) a dual-optics having a basic correction power given by the sum of the front and back optics power;
(b) a pair of haptics and hinge portion,
wherein said lens is implanted in the capsular bag of the eye and said front-optics and said back-optics are allowed to move along the axial direction in reaction to the ciliary-body contraction.

2. An intraocular lens of claim 1, wherein said correction power is about 15 to 25 diopters pre-determined by the measured ocular parameters such that the eye after the lens implant is piano for distance.

3. An intraocular lens of claim 1, wherein said front-optics power is at least +15 diopter and preferably about 30 to 40 diopters.

4. An intraocular lens of claim 1, wherein said back-optics power is less than −2.0 diopters and preferably about −10 to −20 diopter.

5. An intraocular lens of claim 1, wherein said front-optics moves toward the cornea, whereas said back-optics is immobile or moves away from the cornea.

6. An intraocular lens of claim 1, wherein positive accommodation amplitude at least 3.5 diopter, preferably 4.0 to 10.0 diopter is achieved by the change of the spacing between said front and back optics.

7. An intraocular lens of claim 1, wherein adult eyes after cataract surgery is capable of seeing both near and far via the positive accommodation.

8. An intraocular lens of claim 1, wherein said front-optics power is less than −5 diopter, preferably about −10 to −30 diopters and moves toward the cornea resulting from the ciliary body contraction.

9. An intraocular lens of claim 1, wherein said back-optics power is at least +15 diopters, preferably about +20 to +40 diopter and stays immobile or moves away from the cornea resulting from the ciliary body contraction.

10. An intraocular lens of claim 1, wherein negative accommodation amplitude at least −3.0 diopter preferably −4.0 to −6.0 diopter is achieved by the change of the spacing between the said front and back optics.

11. An intraocular lens of claim 1, wherein pediatric eyes after cataract surgery is capable of seeing both near and far via the negative accommodation.

12. A method of providing accommodation for adult and pediatric eyes after cataract surgery comprising of:
(a) selecting a dual-optics intraocular lens having a pre-determined basic power of about 15 to 25 diopter;
(b) implanting said lens within the capsular bag of the eye, wherein accommodation is achieved by the axial movement of the dual-optics in reaction to the ciliary-body contraction.

13. A method of claim 12, wherein said lens consists of a positive power front-optics (at least 15 diopter) and a negative power back-optics (less than −2.0 diopter).

14. A method of claim 13, wherein said front-optics moves toward the cornea, whereas the back-optics moves away from the cornea to produce a positive accommodation for an adult eye to see near.

15. A method of claim 12, wherein said lens consists of a positive power back-optics (at least 15 diopter) and a negative power front-optics (less than −5.0 diopter).

16. A method of claim 15, wherein said front-optics moves toward the cornea whereas the back-optics moves away from the cornea to produce a negative accommodation for a pediatric eye to see near.
